(19) United States
(12) Patent Application Publication
(10) Pub. No.: US 2008/0245692 A1
(43) Pub. Date: Oct. 9, 2008

(54) Carrying assembly with analyte-determining instrument

(75) Inventor: Julianne M. Guarino, Edwardsburg, MI (US)

Correspondence Address: NIXON PEABODY LLP, 161 N. CLARK STREET, 48TH FLOOR, CHICAGO, IL, 60601, US

(73) Assignee: Bayer HealthCare LLC

(21) Appl. No.: 12/079,398

(22) Filed: Mar. 26, 2008

Related U.S. Application Data
(60) Provisional application No. 60/921,942, filed on Apr. 5, 2007.

(51) Int. Cl. A45C 15/00; A45C 13/02; A61B 5/05

(52) U.S. Cl. 206/350; 206/363; 600/345

(57) ABSTRACT

A combination of carrying assembly with analyte-determining device. The analyte-determining device includes housing, a display and at least one user-input mechanism. The carrying assembly is adapted to magnetically attach the analyte-determining device. One of the analyte-determining device and the carrying assembly includes at least one magnet. The other of the analyte-determining device and the carrying assembly includes a magnetic-attracting portion. The at least one magnet is attracted to the at least one magnetic-attracting portion and attaches the analyte-determining device and the carrying assembly with each other.

### CROSS REFERENCE TO RELATED APPLICATION

This application claims priority to Application No. 60/921,942 filed on Apr. 5, 2007, which is incorporated by reference in its entirety.

### FIELD OF THE INVENTION

The present invention relates generally to an analyte-determining device and, more specifically, a carrying assembly with analyte-determining instrument.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests may be used to determine what, if any, insulin or other medication needs to be administered.

To determine the concentration of an analyte in a body fluid, an analyte-determining instrument or meter is typically used. To assist in carrying and protecting this instrument and other devices (e.g., a lancing device, lancets and test sensors), a carrying assembly (e.g., a billfold) may be used. Some existing billfolds include elastic straps that hold the analyte-determining instrument and other devices (e.g., a lancing device). Other existing billfolds use pockets to slidably receive an analyte-determining instrument and other devices. There are disadvantages to such existing billfolds. For example, the billfolds may be difficult for certain individuals who have dexterity problems to remove and replace the analyte-determining instrument and any other devices contained within the billfold. Additionally, if the elastic in the billfold stretches, the instrument or other devices may be inadvertently dropped from the billfold, which can result in damaged, unusable or lost instruments/devices. Also, the pocketed billfold increases the likelihood of the analyte-determining instrument or other devices falling out of the pocket, which can also result in damaged, unusable or lost instruments/devices.

It would be desirable to have a carrying assembly with analyte-determining instrument that overcomes one or more of these disadvantages.

### SUMMARY OF THE INVENTION

According to one embodiment, a combination of carrying assembly with analyte-determining device is disclosed. The analyte-determining device includes housing, a display and at least one user-input mechanism. The carrying assembly is adapted to magnetically attach the analyte-determining device. One of the analyte-determining device and the carrying assembly includes at least one magnet. The other of the analyte-determining device and the carrying assembly includes at least one magnetic-attracting portion. The at least one magnet is attracted to the at least one magnetic-attracting portion and attaches the analyte-determining device and the carrying assembly with each other.

According to one method, a carrying assembly is used in combination to an analyte-determining device. An analyte-determining device is provided that includes housing, a display and at least one user-input mechanism. A carrying assembly is adapted to magnetically secure the analyte-determining device is provided. One of the analyte-determining device and the carrying assembly includes at least one magnet. The other of the analyte-determining device and the carrying assembly includes at least one magnetic-attracting portion. The analyte-determining device is placed into the carrying assembly such that the at least one magnet is attracted to the at least one magnetic-attracting portion and attaches the analyte-determining device and the carrying assembly with each other.

According to another embodiment, a combination of carrying assembly with analyte-determining device is disclosed. The analyte-determining device includes housing, a display and at least one user-input mechanism. A removable pouch is adapted to magnetically secure the analyte-determining device. The carrying assembly is adapted to magnetically secure the removable pouch. The analyte-determining device and the removable pouch includes at least one magnet. The other of the analyte-determining device and the removable pouch includes at least one magnetic-attracting portion. The at least one magnet is attracted to the at least one magnetic-attracting portion and attaches the analyte-determining device and the removable pouch with each other. One of the carrying assembly and the removable pouch includes at least one magnet. The other of the carrying assembly and the removable pouch includes the at least one magnetic-attracting portion. The at least one magnet is attracted to the at least one magnetic-attracting portion and attaches the carrying member and the removable pouch with each other.

### BRIEF DESCRIPTION OF THE DRAWINGS

FIG. 1a is a front view of an analyte-determining instrument according to one embodiment.

FIG. 1b is a back view of the analyte-determining instrument of FIG. 1a.

FIG. 2 is a front view of a carrying member in an open position according to one embodiment.

FIG. 3a is a front view of a lancing device according to one embodiment.

FIG. 3b is a back view of the lancing device of FIG. 3a.

FIG. 4 is a front view of the carrying member of FIG. 2, the analyte-determining instrument of FIGS. 1a, 1b and the lancing device of FIGS. 3a, 3b in which the analyte-dispensing instrument and the lancing device are attached to the carrying member.

FIG. 5a is a front view of an analyte-determining instrument according to another embodiment.

FIG. 5b is a back view of the analyte-determining instrument of FIG. 5a.

FIG. 6 is a front view of a carrying member in an open position according to another embodiment.

FIG. 7 is a front view of the carrying member of FIG. 6, the analyte-determining instrument of FIGS. 5a, 5b in which the analyte-dispensing instrument is attached to the carrying member.

FIG. 8a is a front view of a lancing device according to another embodiment.

FIG. 8b is a back view of the lancing device of FIG. 8a.

FIG. 9 is a front view of the analyte-determining instrument of FIGS. 1a, 1b being inserted into a removable pouch according to one embodiment.

FIG. 10 is a front view of a carrying member according to another embodiment.

FIG. 11 is a front view of the analyte-determining instrument in the pouch of FIG. 9 being attached to the carrying member of FIG. 10.

FIG. 12 is a front view of the analyte-determining instrument of FIGS. 1a, 1b being inserted into a removable pouch according to another embodiment.

FIG. 13 is a front view of a carrying member according to another embodiment.

FIG. 14 is a front view of the pouch of FIG. 12 being attached to the carrying member of FIG. 13 in the absence of an analyte-dispensing instrument.

FIG. 15 is a front view of a carrying member in an open position including a zipper fastener according to one embodiment.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATED EMBODIMENTS

The present invention relates generally to a combination of carrying assembly with analyte-determining instrument or meter. The analyte-determining instrument or meter assists in determining the concentration of the desired analyte. The carrying assembly is adapted to secure and retain the analyte-determining device.

Examples of the types of analytes that may be collected include glucose, lipid profiles (e.g., cholesterol, triglycerides, LDL and HDL), microalbumin, hemoglobin A_{1C}, fructose, lactate, or bilirubin. It is contemplated that other analyte concentrations may also be determined. The analytes may be in, for example, a whole blood sample, a blood serum sample, a blood plasma sample, other body fluids like ISF (interstitial fluid) and urine, and non-body fluids. As used within this application, the term “concentration” refers to an analyte concentration, activity (e.g., enzymes and electrolytes), titers (e.g., antibodies), or any other measure concentration used to measure the desired analyte.

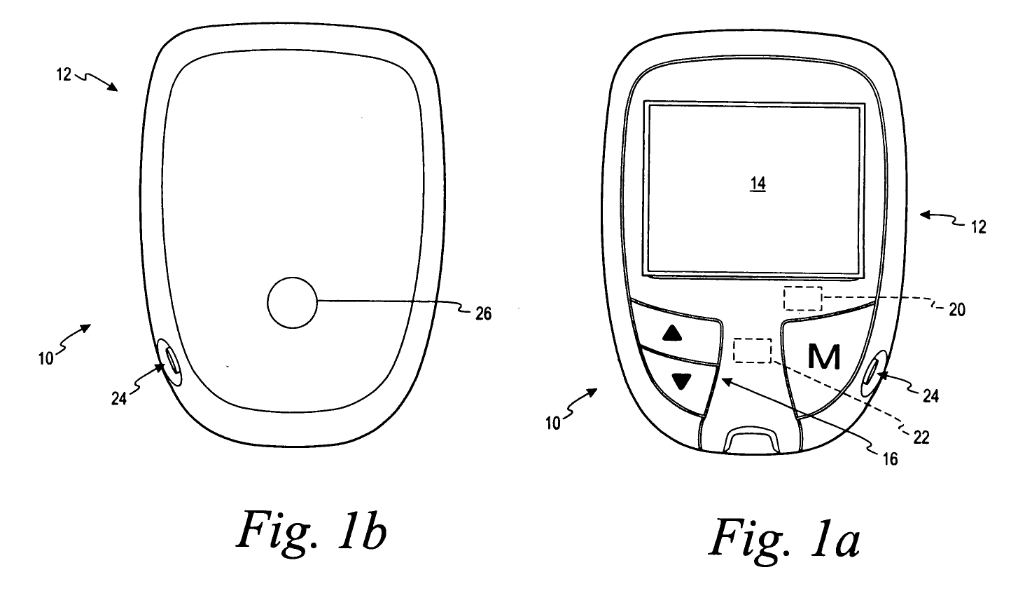

Referring to FIGS. 1a, 1b, an analyte-determining instrument or meter 10 is shown according to one embodiment. The analyte-determining instrument may be a single-sensor meter in one embodiment. In another embodiment, the analyte-determining instrument is a multiple-sensor meter. In one embodiment, the analyte-determining instrument 10 comprises a housing 12, a display 14 and at least one user-input mechanism 16. The analyte-determining instrument 10 is adapted to be secured to a carrying member as will be discussed below. The housing 12 is adapted to assist in protecting the components of the analyte-determining instrument 10. The display 14 is adapted to display information to a user of the instrument 10. Some of the information that may be displayed to a user includes analyte-concentration readings, time and date indicators, hematocrit readings, markers, alarms and any combinations thereof. It is contemplated that other information may be displayed. The display may include different types of displays. For example, the display 14 may include an LCD display, a graphics display, a plasma display, a backlit display, a combination segmented/graphic display or any other suitable display.

The at least one user-input mechanism 16 is adapted to allow the user to make selections relating to one or more user features of the instrument 10. It is also contemplated that the user-input mechanism may allow an individual to turn selected readings to “on” or “off” positions such as analyte concentration, hematocrit readings, or other readings. The user-input mechanism 16 may include, for example, buttons, scroll bars, touch screens, or any combination of such items.

The instrument 10 typically includes a memory device 20 and a processor 22. The memory device 20 is adapted to store analyte concentration readings, hematocrit readings, etc. The processor 22 is adapted to process information and communicate with the memory device 20. The instrument 10 also further includes a data port 24 that is adapted to communicate with a remote device (e.g., a computer) via a communications link. The communications link may be a wired system or wireless.

The housing 12 of FIGS. 1a, 1b in one embodiment is made of polymeric material and includes a magnetic-attracting portion 26. The magnetic-attracting portion 26 is a portion formed in the housing or attached to the housing. The magnetic-attracting portion 26 in one embodiment is a metal portion. The magnetic-attracting portion 26 is shown in FIG. 1b as being generally circular in nature. The magnetic-attracting portion may be of a different size and/or shape than depicted in FIG. 1b. Non-limiting examples of metal that may be used to form the magnetic-attracting portion 26 include iron or steel. It is contemplated that the analyte-determining instrument may include other material besides metal that is adapted to attract to the magnet in the carrying member in this embodiment.

Some commercially available instruments or meters, such as those that are manufactured and/or sold by Bayer Healthcare LLC of Tarrytown, N.Y., may be designed to incorporate the features of the present invention, such as the Ascensia® CONTOUR® Blood Glucose Monitoring System, the Ascensia® BREEZE® and BREEZE®2 Blood Glucose Monitoring System, and the Ascensia® Elite® and Elite® XL Blood Glucose Monitoring System. It is contemplated that other instruments or meters, in addition to the ones listed above, may be designed to incorporate the features of the present invention.

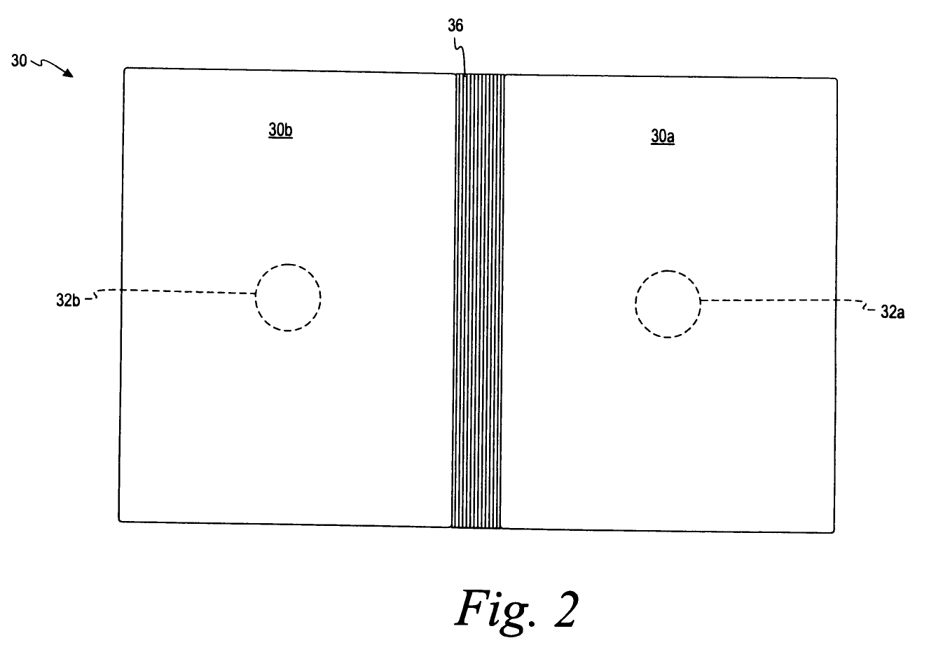

Referring to FIG. 2, a carrying member 30 according to one embodiment is shown that is adapted to assist in protecting and carrying the analyte-determining instrument 10. In one embodiment, the carrying member 30 includes a plurality of magnets 32a, 32b. The magnets 32a, 32b of the carrying member 30 attract the magnet-attracting portion 26 of the analyte-determining instrument 10 (see also FIG. 1b). The magnets 32a, 32b may be any suitable magnet that is of sufficient strength to attract, secure and hold the analyte-determining instrument 10. The magnetic force between the magnetic-attracting portion 26 of the analyte-determining instrument 10 and the magnets 32a, 32b of the carrying member 30 is sufficiently high such that the analyte-determining instrument 10 is securely attached to the carrying member 30, but not too strong as to inhibit removal by a user.

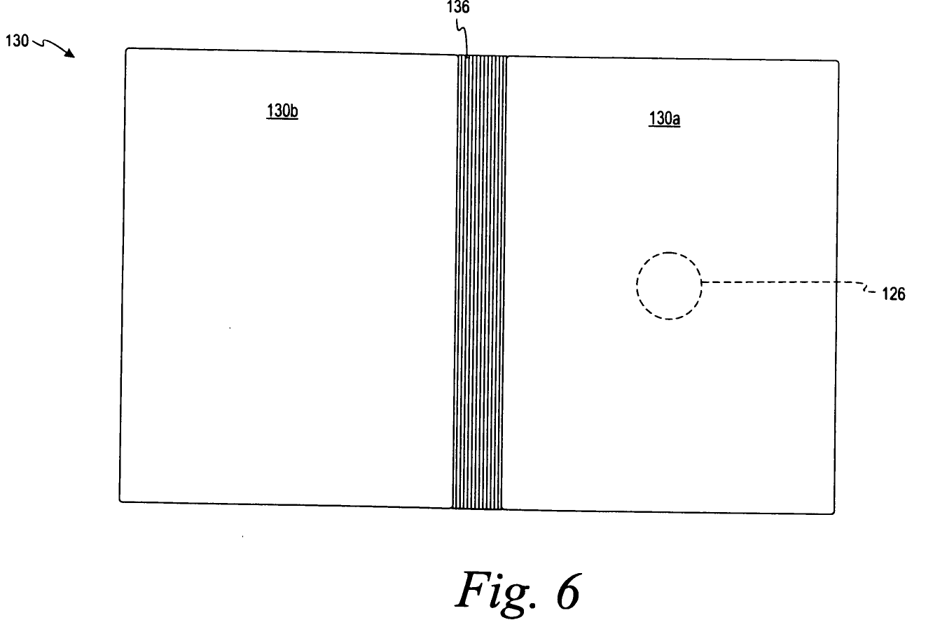

The magnets 32a, 32b may be located in different areas in the carrying member 30. For example, the magnets 32a, 32b may be located within the material that forms the carrying member 30 as shown in FIG. 2. In such a location, the magnets may be sewed, adhered or otherwise attached on the inside of the material that forms the carrying member. In this embodiment, the magnets are located between an outside material and an inside material. According to another embodiment, the magnets may also be secured on an outer surface of the material that forms the carrying member. The magnets 32a, 32b are shown as being circular in FIG. 2. It is contemplated that the magnets 32a, 32b may be of other polygonal or non-polygonal shapes and/or different sizes. It is contemplated that the carrying member in a further embodiment may include exactly one magnet, as will be discussed in connection with FIG. 6 below.

The carrying member 30 of FIG. 2 is shown as a billfold. The carrying member 30 has a first portion 30a and a second portion 30b separated by at least one fold line or crease 36. A billfold is a desirable embodiment because it is foldable and generally compact such that a user can store the carrying member in convenient locations. The analyte-determining instrument may remain in the carrying member while in use. It is contemplated that the carrying member may be of other types. For example, the carrying member may be in the form of a planner or clip that attaches to a belt.

The carrying member 30 may be formed of different materials such as a polymeric material (e.g., nylon or vinyl), a woven fabric, a non-woven fabric and leather. It is contemplated that the carrying member may be made of different types of materials.

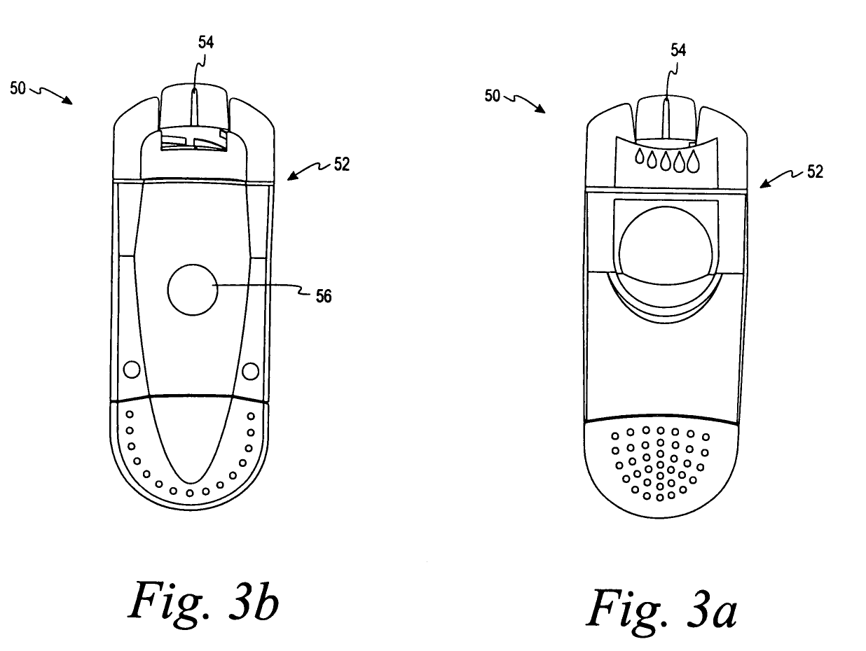

Besides assisting in protecting and carrying the analyte-determining instrument 10, the carrying member 30 in one embodiment may be used to attract, secure, protect and carry other items. For example, one of the magnets 32a, 32b of the carrying member 30 may be used to secure other items. One such item is a lancing device (e.g., lancing device 50 of FIGS. 3a, 3b). It is contemplated that other lancing devices may be used. The lancing device 50 of FIGS. 3a, 3b includes housing 52, a lancet 54 and a magnetic-attracting portion 56 (FIG. 3b) that attracts to one of the magnets 32a, 32b of the carrying member 30. The lancing device 50 is adapted to move the lancet 54 from a retracted position to a lancing position that is adapted to draw fluid. The magnetic-attracting portion 56 may be made of a metal such as those discussed above in forming the magnetic-attracting portion 26. The magnetic-attracting portion 56 may be located on the back of the housing 52 in one embodiment as shown in FIG. 3b. In another embodiment, a magnetic-attracting portion of a lancing device may be located within the housing 52.

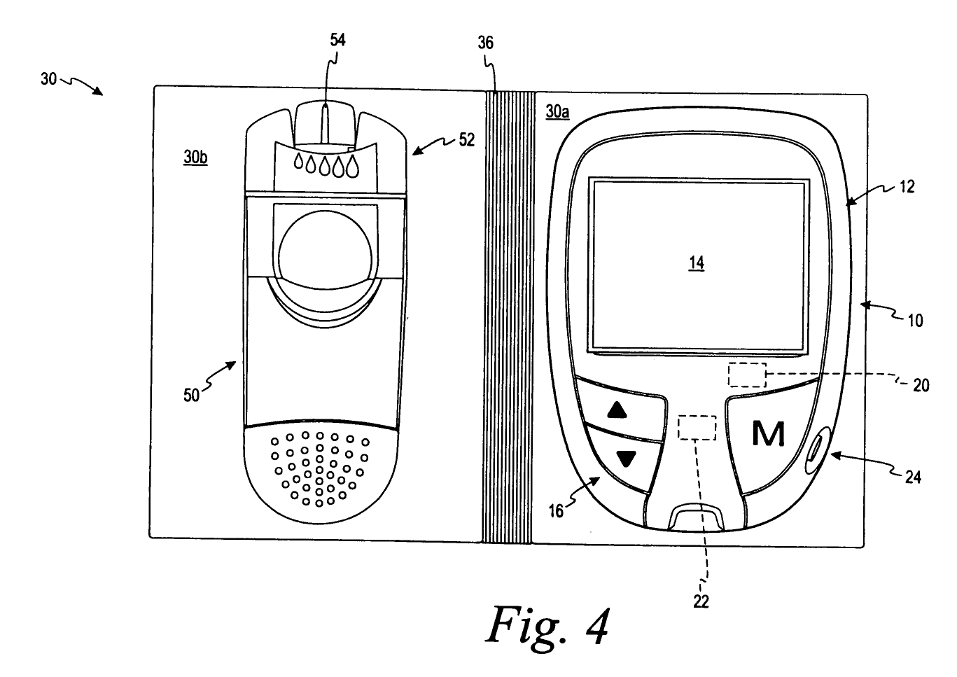

Referring to FIG. 4, the carrying member 30 includes analyte-determining instrument 10 and the lancing device 50. Specifically, magnet 32a attracts the magnetic-attracting portion 26 of the analyte-determining instrument 10 and magnet 32b attracts the magnetic-attracting portion 56 of the lancing device 50.

In addition to the instrument 10 and the lancing device 50, the carrying member 30 may be adapted to secure and carry other supplies such as test strips, lancets, notes, instructions, writing instruments, cotton balls, bandages and/or a log book.

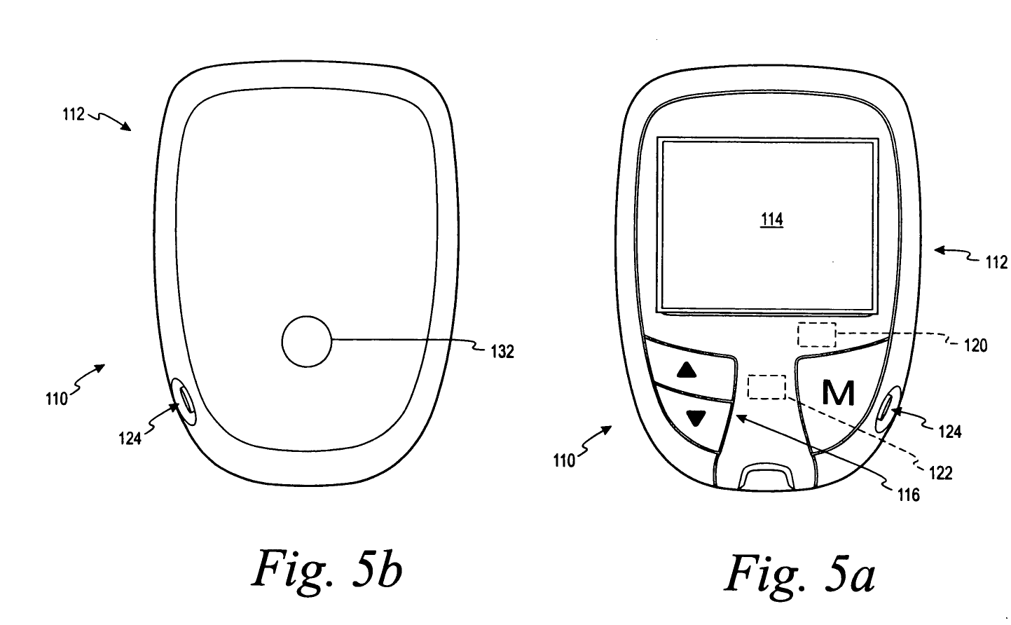

Referring to FIGS. 5a, 5b, an analyte-determining instrument or meter 110 is shown according to another embodiment. The analyte-determining instrument 110 comprises a housing 112, a display 114, at least one user-input mechanism 116, a memory device 120, a processor 122 and a data port 124.

The housing 112 of FIGS. 5a, 5b in one embodiment is made of polymeric material and includes a magnet 132. The magnet 132 may be located on an exterior surface of the housing 112 such as shown in FIG. 5b. In another embodiment, the magnet may be formed as a portion of the exterior surface. Alternatively, the magnet may be located within the housing 112. The magnet may be of a different size and/or shape than depicted in FIG. 5b. The magnet 132 attracts a magnet-attracting portion 126 of a carrying member 130 (FIG. 6). The magnet 132 may be any suitable magnet that is of sufficient strength to attract, secure and hold to the magnetic-attracting portion 126. The magnetic force between the magnetic-attracting portion 126 and the magnet 132 is sufficiently high such that the analyte-determining instrument 110 is securely attached to the carrying member 130, but not too strong as to inhibit removal by a user.

The magnetic-attracting portion 126 in one embodiment is a metal portion. The magnetic-attracting portion 126 is shown in FIG. 6 as being generally circular in nature. The magnetic-attracting portion may be of a different size and/or shape than depicted in FIG. 6. Non-limiting examples of metal that may be used to form the magnetic-attracting portion 126 include iron or steel. It is contemplated that the magnetic-attracting portion of the carrying member may include other material besides metal that is adapted to attract to the magnet in the analyte-determining instrument.

The carrying member 130 of FIG. 6 is in the form of a billfold. The carrying member 130 includes a first portion 130a and a second portion 130b separated by at least one fold line or crease 136.

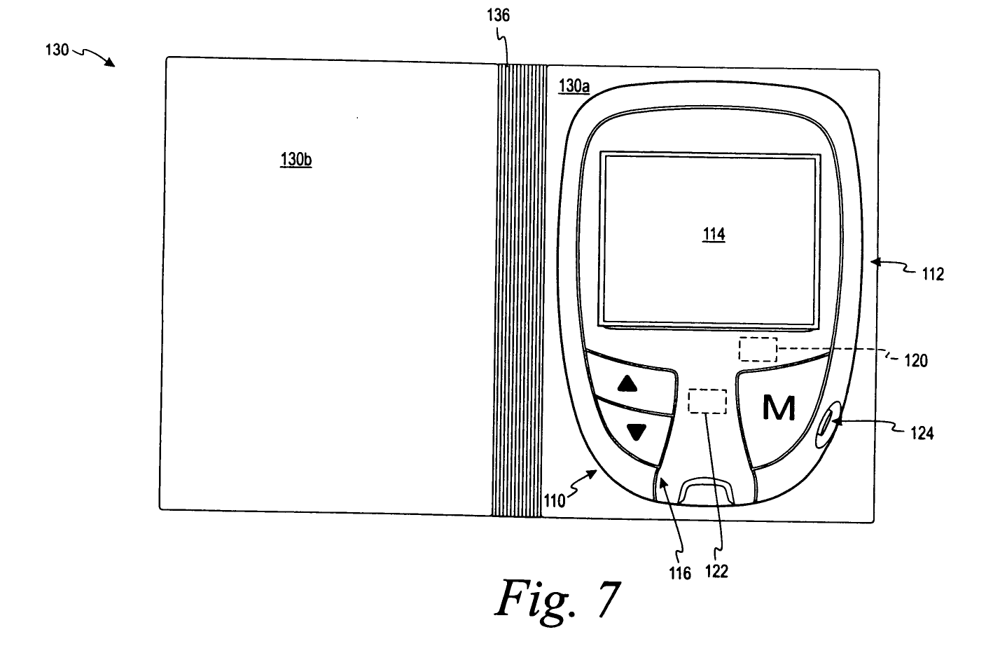

The magnetic-attracting portion 126 may be located in different areas in the carrying member 130. For example, the magnetic-attracting portion 126 may be located within the material that forms the carrying member 130 as shown in FIG. 6. In such a location, the magnetic-attracting portion may be sewed, adhered or otherwise attached on the inside of the material that forms the carrying member. In this embodiment, the magnetic-attracting portion is located between an outside material and an inside material. The magnetic-attracting portion may also be secured on an outer surface of the material that forms the carrying member according to another embodiment. The carrying member 130 is shown with attached analyte-dispensing instrument 110 thereto in FIG. 7.

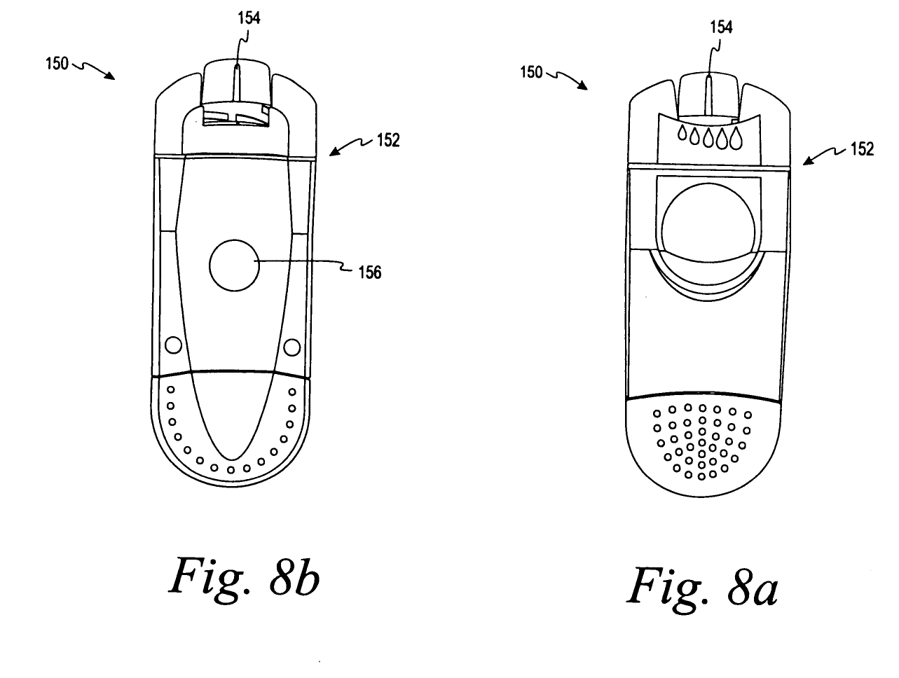

It is contemplated that the magnetic-attracting portion 126 (FIG. 6) may be used to assist in securing other items such a lancing device with a magnet. Such a lancing device is shown in FIG. 8a, 8b with lancing device 150. The lancing device 150 includes housing 152, lancet 154 and a magnet 156. The magnet 156 of the lancet is adapted to attract and be secured with the magnetic-attracting portion 126 of the carrying member 130.

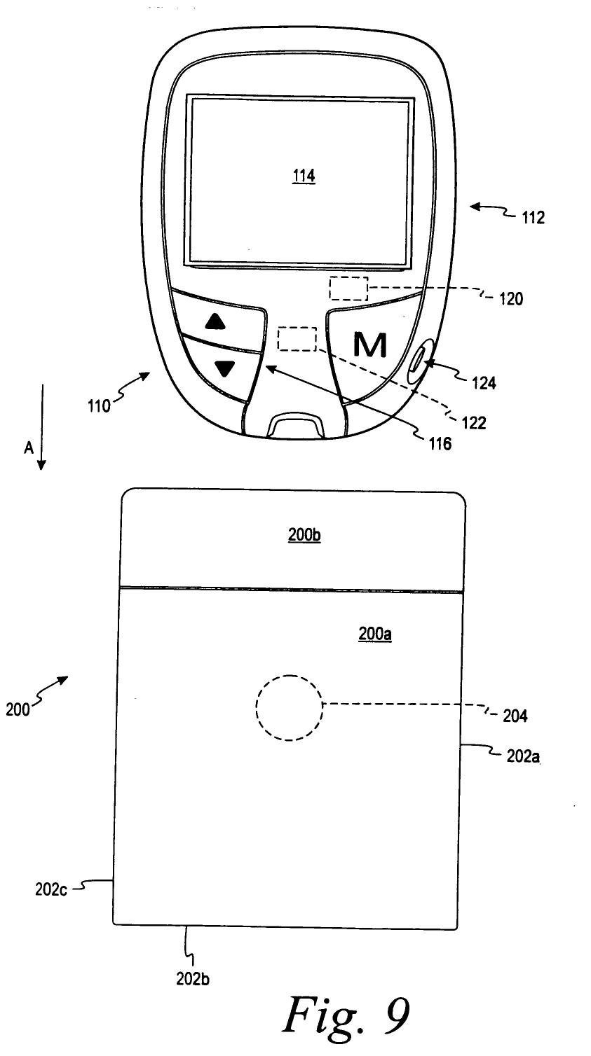

Referring to FIGS. 9-11, a removable pouch embodiment is shown. Referring initially to FIG. 9, the analyte-determining instrument 110 is shown with a removable pouch 200. The removable pouch 200 is formed of two portions 200a, 200b that are attached together along edges 202a, 202b and 202c. The edges may be secured by any suitable method such as adhesive or a mechanical method such as sewing. One of the edges of the removable pouch 200 may be formed by folding a continuous sheet into two portions 200a, 200b. The opposing side from edge 202b is open and is sized to receive the analyte-determining instrument 110. More specifically, as shown in FIG. 9, the analyte-determining instrument 110 may be slid generally along the direction of arrow A into the removable pouch 200.

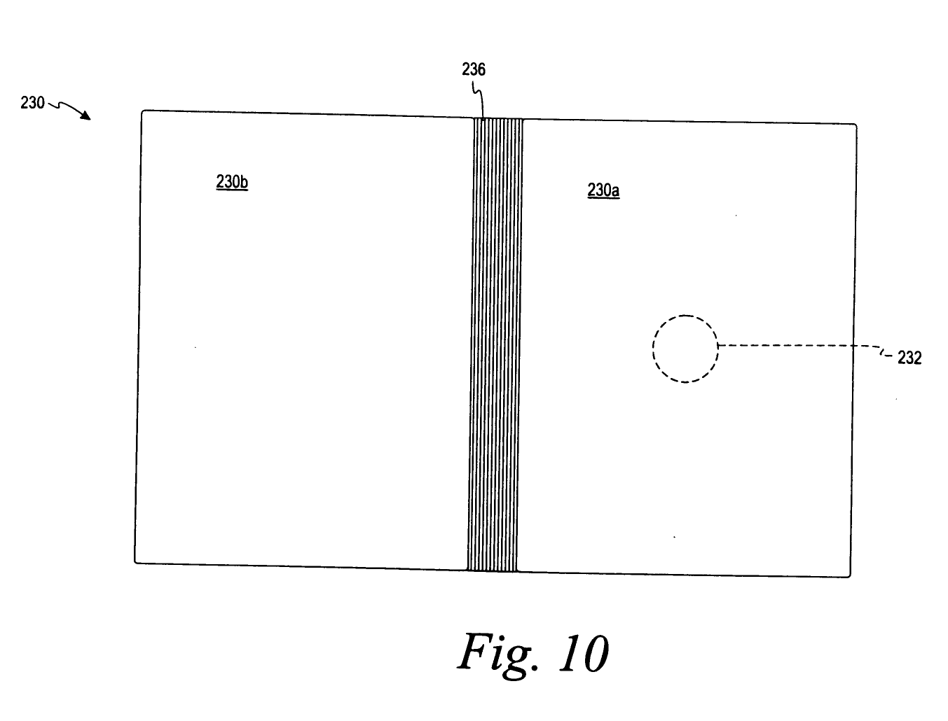

The removable pouch 200 includes a magnetic-attracting portion 204 that is adapted to be secured to a magnet. A magnet 232 of a carrying member 230 as shown in FIG. 10 is adapted to attract, secure and hold the magnetic-attracting portion 204 of the removable pouch 200. The magnetic-attracting portion 204 is adapted to be attracted to the magnet 232. The magnet 232 may be located within the carrying member in a similar manner as discussed above with respect to the magnets 32a, b of the carrying member 30.

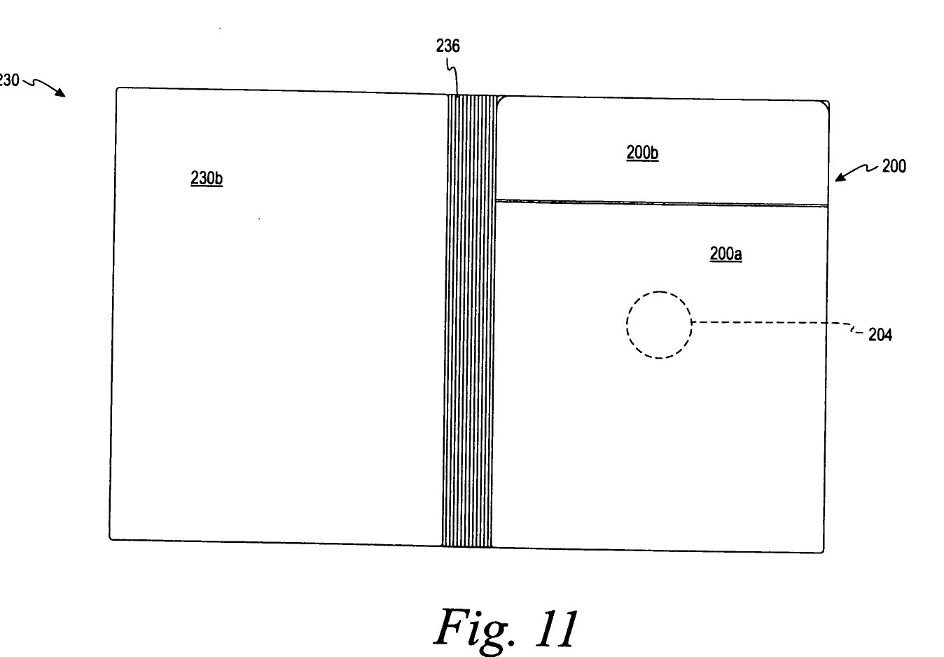

Referring to FIG. 11, the carrying member 230 includes the removable pouch 200. Specifically, magnet 232 attracts the magnetic-attracting portion 204 of the removable pouch 200.

It is contemplated that the removable pouch 200 may be used with the lancing device 50. In this embodiment, the lancing device 50 would be slid into the removable pouch 200 in a similar manner as discussed above with respect to the analyte-determining instrument 110.

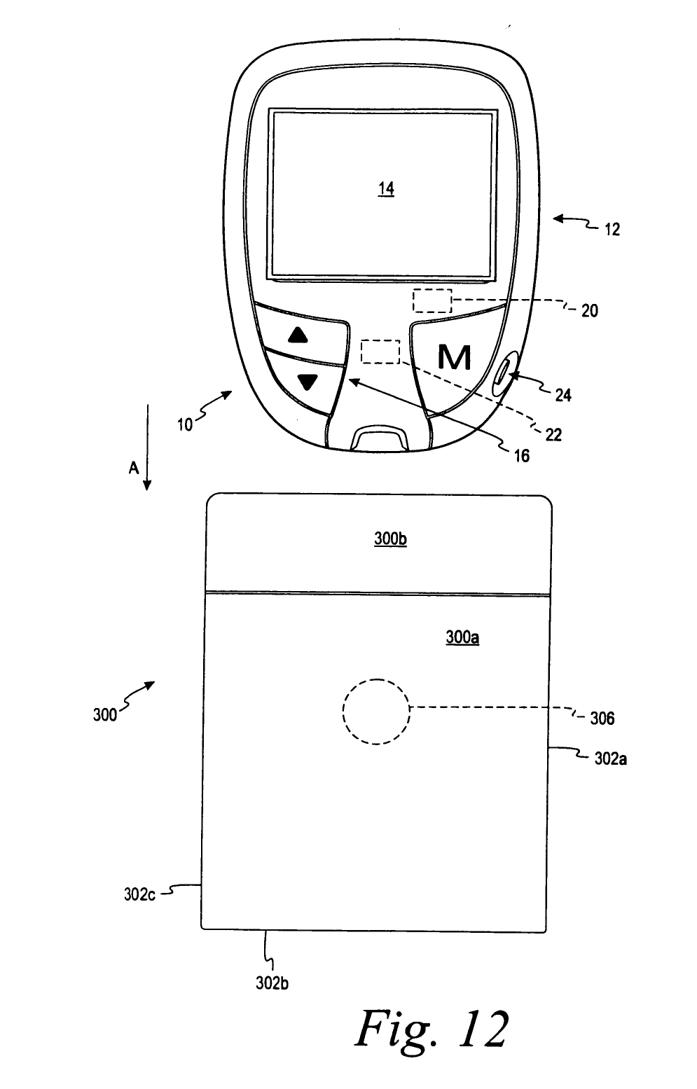

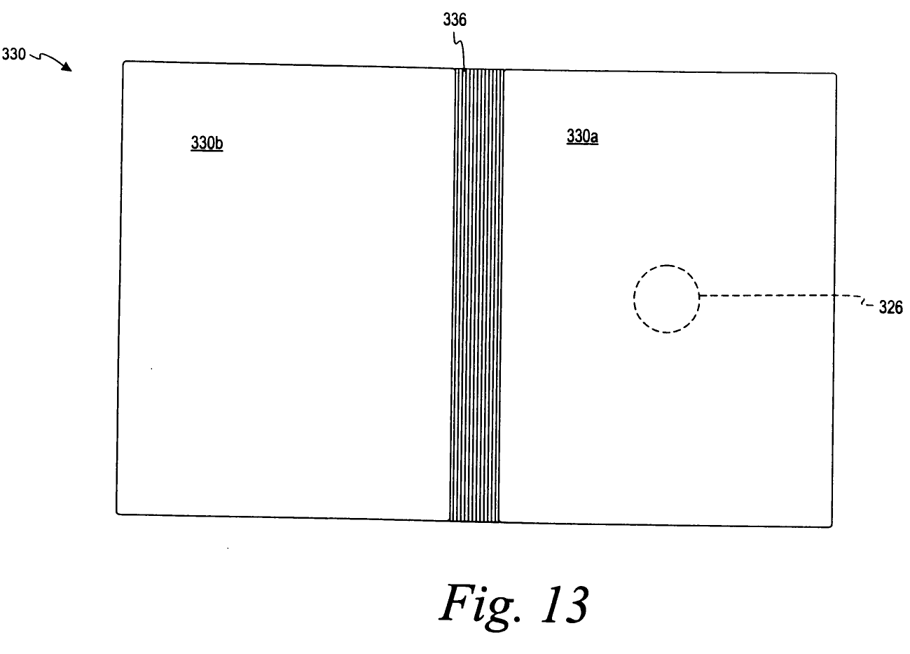

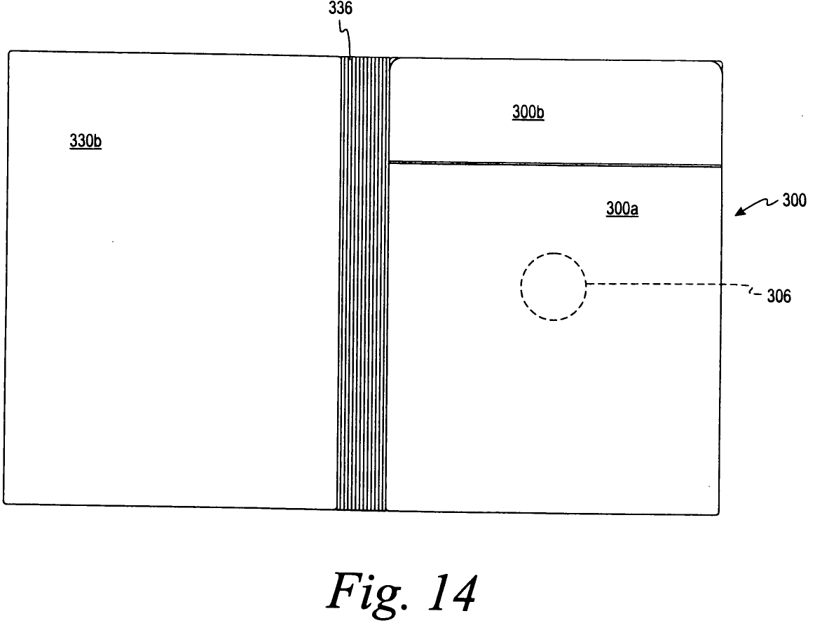

In another embodiment, a removable pouch 300 is shown in FIG. 12; The removable pouch 300 is formed of two portions 300a, 300b that are attached together along edges 302a, 302b and 302c. The removable pouch 300 functions in a similar manner as removable pouch 200 except that the removable pouch includes a magnet 306, while the removable pouch 200 includes the magnetic-attracting portion 204. The magnet 306 of the removable pouch 300 is adapted to be secured to a magnetic-attracting portion 326 of a carrying member 330 (see FIGS. 13 and 14). The carrying member 330 includes a first portion 330a and a second portion 330b.

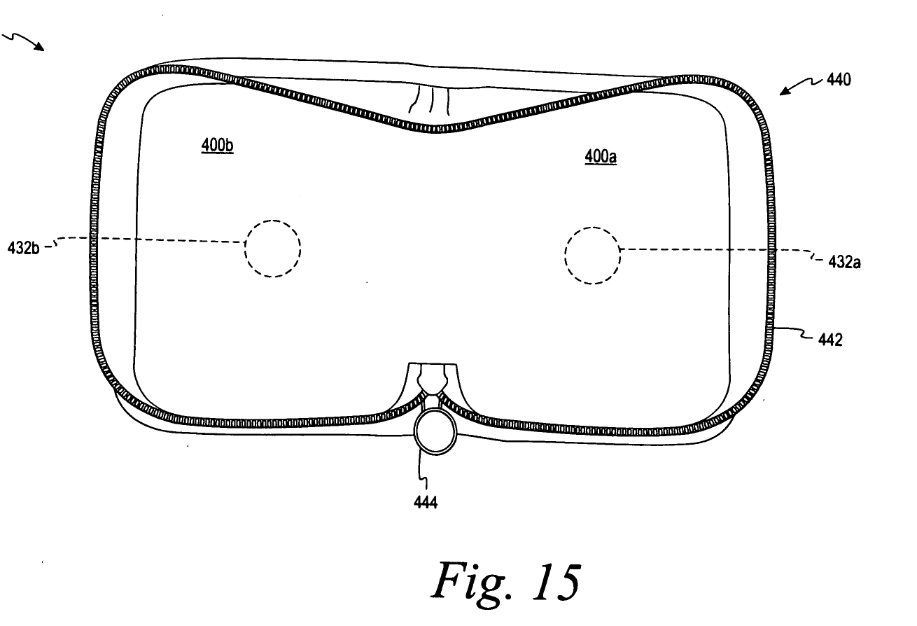

It is contemplated that the carrying members may be different than shown in FIGS. 2, 6 and 10. For example, FIG. 15 shows a carrying member 400 with a first portion 400a and a second portion 400b. Each of the first and second portions 400a, , 400b includes a respective magnetic 432a, 432b. As shown in FIG. 15, the carrying member 400 includes a zipper fastener 440 in an open position. The zipper fastener 440 includes track 442 and a zipper 444. When the zipper 444 is moved along track 442 to a closed position, the first and second portions 400a, 400b generally overlap each other.

In one method, an individual opens a carrying member (e.g., billfold) and removes the analyte-determining instrument from the billfold. More specifically, the analyte-determining instrument is pulled with sufficient force to overcome the magnetic attraction between the analyte-determining instrument and the billfold. If additional items are contained within the carrying member (e.g., lancing device), they may be removed in a similar manner by pulling the additional items from the billfold. The analyte-determining instrument and any additional items may be returned to the carrying member by reestablishing the magnetic connection. The analyte-determining instrument is typically removed from the carrying member to perform the testing of the analyte concentration of the fluid. It is contemplated that the testing of the analyte concentration of the fluid may be performed without removing the analyte-determining instrument from the carrying member.

What is claimed is:

1. A combination of carrying assembly with analyte-determining device, the combination comprising:
an analyte-determining device including housing, a display and at least one user-input mechanism; and
a carrying assembly being adapted to magnetically attach the analyte-determining device,
wherein one of the analyte-determining device and the carrying assembly includes at least one magnet, the other of the analyte-determining device and the carrying assembly including at least one magnetic-attracting portion, the at least one magnet being attracted to the at least one magnetic-attracting portion and attaching the analyte-determining device and the carrying assembly with each other.

2. The combination of claim 1, wherein the carrying member is a billfold.

3. The combination of claim 1, wherein the carrying assembly includes the at least one magnet and the analyte-determining device includes the at least one magnetic-attracting portion.

4. The combination of claim 3, wherein the at least one magnet is located within the material that forms the carrying assembly.

5. The combination of claim 1, wherein the analyte-determining device includes the at least one magnet and the carrying member includes the at least one magnetic-attracting portion.

6. The combination of claim 1, further including a lancing device and wherein at least one of the lancing device and the carrying assembly includes at least one magnet, the other of the lancing device and the carrying assembly including the at least one magnetic-attracting portion, the at least one magnet being attracted to the at least one magnetic-attracting portion and attaching the lancing device and the carrying assembly with each other.

7. A method of using a carrying assembly and an analyte-determining device, the method comprising the acts of:
providing an analyte-determining device including housing, a display and at least one user-input mechanism;
providing a carrying assembly being adapted to magnetically secure the analyte-determining device, wherein one of the analyte-determining device and the carrying assembly includes at least one magnet, the other of the analyte-determining device and the carrying assembly including at least one magnetic-attracting portion; and
placing the analyte-determining device into the carrying assembly such that the at least one magnet is attracted to the at least one magnetic-attracting portion and attaches the analyte-determining device and the carrying assembly with each other.

8. A combination of carrying assembly with analyte-determining device, the combination comprising:
an analyte-determining device including housing, a display and at least one user-input mechanism;
a removable pouch being adapted to magnetically secure the analyte-determining device; and
a carrying assembly being adapted to magnetically secure the removable pouch,
wherein the analyte-determining device and the removable pouch includes at least one magnet, the other of the analyte-determining device and the removable pouch including at least one magnetic-attracting portion, the at least one magnet being attracted to the at least one magnetic-attracting portion and attaching the analyte-determining device and the removable pouch with each other,
wherein one of the carrying assembly and the removable pouch includes at least one magnet, the other of the carrying assembly and the removable pouch including the at least one magnetic-attracting portion, the at least one magnet being attracted to the at least one magnetic-attracting portion and attaching the carrying member and the removable pouch with each other.

9. The combination of claim 8, wherein the carrying member is a billfold.

10. The combination of claim 8, wherein the carrying assembly includes the at least one magnet, the analyte-dispensing instrument including the at least one magnet, the removable pouch including the at least one magnetic-attracting portion.

11. The combination of claim 10, wherein the magnet is located within the material that forms the carrying assembly.

12. The combination of claim 8, wherein the carrying assembly includes the at least one magnet-attracting portion, the analyte-dispensing instrument including the at least one magnetic-attracting portion, the removable pouch including the at least one magnet.

13. The combination of claim 8, wherein the carrying member is a billfold.
While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention.
